(19) United States
(12) Patent Application Publication
(10) Pub. No.: US 2008/0262114 A1
(43) Pub. Date: Oct. 23, 2008

(54) FISCHER-TROPSCH CATALYST SUPPORT AND CATALYST

(76) Inventor: Marinus Johannes REYNHOUT, Amsterdam (NL)

Correspondence Address: SHELL OIL COMPANY, P O BOX 2463, HOUSTON, TX, 772522463, US

(21) Appl. No.: 12/016,699

(22) Filed: Jan. 18, 2008

(30) Foreign Application Priority Data
Jan. 18, 2007 (EP) 07100754.6

(51) Int. Cl. C07C 1/04; B01J 32/00; B01J 21/06; B01J 23/10; B01J 23/16; B01J 23/32; B01J 23/40; B01J 23/48; B01J 23/72; B01J 23/75; B01J 23/755

(52) U.S. Cl. 518/719; 502/439; 502/325; 502/337; 502/338; 518/715; 502/304; 502/302; 502/305; 502/324; 502/353; 502/340; 502/352; 502/349; 502/339; 502/330; 502/240

(57) ABSTRACT

A Fischer-Tropsch catalyst support comprising at least 15 wt % of a material having the formula XₐY_{b}O_{c} wherein: X comprises an element selected from the group consisting of magnesium, calcium, barium, strontium, cerium, vanadium, chromium, manganese, iron, cobalt, nickel, copper, zinc, niobium, ruthenium, rhodium, palladium, cadmium, osmium, iridium, platinum, gold, mercury, tin, lead, lanthanides, and mixtures thereof; Y comprises a different element to X, Y selected from the group consisting of silicon, aluminium, titanium, zirconium, cerium, hafnium, gallium, and mixtures of these, preferably silicon, aluminium and titanium and mixtures thereof, especially titanium; O is oxygen; a and b are, independently, in the range of 1-6; c is in the range of 1-15. Preferably a perovskite-type structure results which is more stable and resistant to degradation.

This application claims the benefit of European Application 07100754.6 filed Jan. 18, 2007.

### BACKGROUND OF THE INVENTION

This invention relates to a catalyst support, catalyst precursor, and catalyst suitable for use in a Fischer-Tropsch reaction.

Many documents are known describing methods and processes for the catalytic conversion of (gaseous) hydrocarbonaceous feedstocks, especially methane, natural gas and/or associated gas, into liquid products, especially methanol and liquid hydrocarbons, particularly paraffinic hydrocarbons.

The Fischer-Tropsch process can be used for the conversion of synthesis gas (from hydrocarbonaceous feed stocks) into liquid and/or solid hydrocarbons. Generally, the feed stock (e.g. natural gas, associated gas and/or coal-bed methane, heavy and/or residual oil fractions, coal, biomass) is converted in a first step into a mixture of hydrogen and carbon monoxide (this mixture is often referred to as synthesis gas or syngas). The synthesis gas is then fed into one or more reactors where it is converted in one or more steps over a suitable catalyst at elevated temperature and pressure into paraffinic compounds ranging from methane to high molecular weight modules comprising up to 200 carbon atoms, or, under particular circumstances, even more. Preferably the amount of C₅₊ hydrocarbons produced is maximized and the amount of methane and carbon dioxide is minimized.

The Fischer-Tropsch process is a catalyzed process.

One preferred catalyst for the process comprises a cobalt active phase supported on a porous refractory oxide such as titania. Manganese or vanadium may be added as promoters. Typically this is prepared by shaping, drying and calcining a titania catalyst precursor and adding the required active metals and optionally promoter before or after the shaping of the titania.

Conventionally, the calcination temperature is preferably maximized in order to provide strength to the catalyst support. However at high temperatures, the active cobalt reacts with the titania support to produce inert cobalt titanate thus rendering active cobalt inactive. Whilst the deactivated catalyst can be removed and reactivated, it is more efficient to prevent the formation of cobalt titanate by imposing an upper limit on the calcination temperature to allow no more than 1 wt % of cobalt titanate to form based on the total catalyst.

Thus the upper limit of the calcination temperature is typically around 550-600° C. to prevent a significant amount of cobalt titanate from forming.

Moreover, whilst the in situ strength of known catalysts is sufficient, they do deteriorate over time due to sintering of the support material. Thus an increased stability of the support material will increase the longevity of the catalysts.

### SUMMARY OF THE INVENTION

According to a first aspect of the present invention there is provided a Fischer-Tropsch catalyst support comprising at least 15 wt % of a material having the formula XₐY_{b}O_{c} wherein:

X comprises an element selected from the group consisting of magnesium, calcium, barium, strontium, cerium, vanadium, chromium, manganese, iron, cobalt, nickel, copper, zinc, niobium, ruthenium, rhodium, palladium, cadmium, osmium, iridium, platinum, gold, mercury, tin, lead, lanthanides, and mixtures thereof;

Y comprises a different element to X selected from the group consisting of silicon, aluminium, titanium, zirconium, cerium, hafnium, gallium and mixtures of these, preferably silicon, aluminium and titanium and mixtures thereof, especially titanium;

O is oxygen;

a and b are, independently, in the range of 1-6;

c is in the range of 1-15.

### DETAILED DESCRIPTION OF THE INVENTION

The inventors of the present invention have found that by using a support material consisting of at least 15% XₐY_{b}O_{c} the stability and strength of the catalyst support material is much improved and the in-situ formation of, for example, cobalt titanate is resisted since the support is more stable thus allowing increased calcination temperatures and associated benefits such as improved hydrothermal stability and other mechanical properties. Moreover the catalyst support material is more resistant to degradation over time.

Certain embodiments of the invention may be calcined at 900° C. without loss of specific surface area, or growth in support particle size, thus producing a catalyst with improved hydrothermal stability and mechanical strength.

Small amounts of related compounds may also be present which have a, b and c values greater than the given range.

Preferably a and b are, independently, in the range of 1-4; more preferably a and b are independently 1 or 2. Preferably c is in the range of 1-9.

Normally X is in a 2+ oxidation state, but it can also be in a 3+ oxidation state. Normally Y is in a 4+ oxidation state but it can also be in a 3+ or in a 5+ oxidation state.

Certain pervoskites have the ilmenite type structure and preferably the present invention has an ilmenite type structure.

Preferably XₐY_{b}O_{c} is in a nano-sized crystalline form preferably of the size of 10-100 nm, preferably 40-60 nm. Preferably it is a porous support material. Alternatively it may be in amorphous form. Preferably the XₐY_{b}O_{c} is formed in the absence of hydrogen especially in the absence of carbon monoxide.

Preferably the XₐY_{b}O_{c} is formed in the absence of water. Optionally the XₐY_{b}O_{c} may be recovered as a mineral deposit.

Thus typically the support consists of more than 15 wt % XₐY_{b}O_{c}, preferably more than 30 wt % XₐY_{b}O_{c}, more preferably more than 50 wt % XₐY_{b}O_{c} and in especially preferred embodiments more than 80 wt % XₐY_{b}O_{c}, even more than 95 wt % XₐY_{b}O_{c}. This is in marked contrast to the teaching in the art where the smallest formation of, for example, cobalt titanate, is not wanted. Indeed those skilled in the art strive to prevent such formation contrary to the teaching herein. All percentages specified in the context refer to weight percent of the total catalyst support, not including therefore catalytically active metals or promoters.

In addition to XₐY_{b}O_{c}, the support may comprise YO₂ wherein Y is as previously defined, for example TiO₂.

The catalyst support may also comprise a material of the formula (XX′)ₐY_{b}O_{c} wherein X, Y, a, b, O and c are as previously defined and X′ is another metal selected from the group defined for X.

Also, XₐY_{b}O_{c} can comprise X_{a′}Y_{b}O_{c} and X″_{a″}Y_{b}O_{c} wherein a′ and a″ are as previously defined for a and X″ is as previously defined for X.

Small amounts of Fe, Co, Ni in particular may be present, preferably Fe when in combination with, for example, Mn as X.

Preferably the element X is one or more selected from the group consisting of: manganese, vanadium, magnesium, calcium, barium, strontium, cerium, cobalt, iron, ruthenium, chromium, tin, lead, palladium, lanthanides, lanthanides, and nickel.

More preferably the element X is one or more selected from the group consisting of: manganese, vanadium, cerium, cobalt, iron, ruthenium and nickel.

For certain embodiments, the element X is a catalytically active Fischer-Tropsch metal such as one or more selected from the group consisting of iron, cobalt, nickel and ruthenium. When the element X is a catalytically active Fischer-Tropsch metal, it preferably is cobalt.

Thus for such embodiments, the element X is unlikely to detrimentally affect the system if it comes out of the support since it is known for X to be used as a catalytically active metal in a Fischer-Tropsch reaction.

For such embodiments, preferably XₐY_{b}O_{c} is in the form of a perovskite-type structure including structures of the general formula (XO)ᵢ(YO₂)ᵢᵢ (wherein (i) and (ii) are arbitrary constants) such as (CoO)(TiO₂), FeO(SiO₂), NiO(SiO₂) and FeO(Al₂O₄).

In other embodiments, the element X is a metal suitable to promote a Fischer-Tropsch reaction such as one or more selected from the group consisting of: magnesium, calcium, barium, strontium, cerium, thorium, uranium, especially zirconium, manganese, vanadium, hafnium, cerium, thorium, uranium; more especially manganese and vanadium. When the element X is a metal suitable to promote a Fischer-Tropsch reaction, it preferably is manganese.

When X is manganese the support may be for example, MnTiO₃, MnAl₂O₄ or MnSiO₃.

Preferably XₐY_{b}O_{c} is in the form of a perovskite-type structure including structures of the general formula (XO)ᵢ(YO₂)ᵢᵢ (wherein (i) and (ii) are arbitrary constants) such as (MnO)ᵢ(TiO₂)ᵢᵢ which includes: Mn₂TiO₄, MnTi₂O₅, MnTi₃O₇, and solid solution FeTi₂O₅—MgTi₂O₅—MnTi₂O₅. The solid solution FeTi₂O₅—MgTi₂O₅—MnTi₂O₅ is sometimes referred to as (Fe, Mg, Mn)Ti₂O₅.

Thus for such embodiments, the element X′ is unlikely to detrimentally affect the system if it comes out of the support since it to encourage (as a promoter) not discourage the Fischer-Tropsch reaction.

A catalytically active metal or precursor therefor may be added onto the support to form a catalyst or catalyst precursor.

Thus the present invention also provides a Fischer-Tropsch catalyst or catalyst precursor comprising:

a catalytically active metal or precursor therefor, whereby the catalytically active metal is selected from the group consisting of: ruthenium, iron, cobalt, ruthenium and nickel or combinations thereof, preferably cobalt;

optionally one or more promoters or precursor(s) therefor;

a Fischer-Tropsch catalyst support as described herein.

For certain embodiments, preferably the support material XₐY_{b}O_{c} comprises the same element (X) as a, or the, catalytically active metal in the catalyst.

For certain embodiments, preferably the support material XₐY_{b}O_{c} comprises the same element (X) as a, or the, promoter in the catalyst.

For embodiments whereby the support material XₐY_{b}O_{c} comprises the same element (X) as either (i) the catalytically active metal in the catalyst or (ii) a or the promoter in the catalyst, the system will not be affected by a foreign element in the event of the element X coming out of the support material, because the same element is already present in the system as an active metal or as a promoter.

Indeed for certain embodiments, no or less separate active metal (or promoter) may be added to the catalyst and a portion of the active metal-type element (or promoter type element) within the support can provide the catalytic (or promotion) properties by coming out of the XₐY_{b}O_{c} structure. This may be encouraged by partially reducing the catalyst to remove some of the active metal type element (or promoter-type element) from the XₐY_{b}O_{c} structure.

The catalytically active material, generally based on a catalytically active metal, may be present with one or more metals or metal oxides as promoters, more particularly one or more d-metals or d-metal oxides.

Suitable metal oxide promoters may be selected from Groups 2-7 of the Periodic Table of Elements, or the actinides and lanthanides. In particular, oxides of magnesium, calcium, strontium, barium, scandium, yttrium, lanthanum, cerium, titanium, zirconium, hafnium, thorium, uranium, vanadium, chromium and manganese are most suitable promoters.

Suitable metal promoters may be selected from Groups 7-10 of the Periodic Table. Manganese, iron, rhenium and Group 8-10 noble metals are particularly suitable, with platinum and palladium being especially preferred. The amount of promoter present in the catalyst is suitably in the range of from 0.01 to 100 pbw, preferably 0.1 to 40, more preferably 1 to 20 pbw, per 100 pbw of support.

References to “Groups” and the Periodic Table as used herein relate to the new IUPAC version of the Periodic Table of Elements such as that described in the 87^{th} Edition of the Handbook of Chemistry and Physics (CRC Press).

The catalytically active material could also be present with one or more co-catalysts. Suitable co-catalysts include one or more metals such as iron, nickel, or one or more noble metals from Groups 8-10. Preferred noble metals are platinum, palladium, rhodium, ruthenium, iridium and osmium. Most preferred co-catalysts for use in the hydro-cracking are those comprising platinum. Such co-catalysts are usually present in small amounts.

A suitable catalyst comprises cobalt as the catalytically active metal and zirconium as a promoter on a support as defined herein.

Another suitable catalyst comprises cobalt as the catalytically active metal and manganese and/or vanadium as a promoter on a support as defined herein.

One particularly preferred Fischer-Tropsch catalyst comprises a cobalt catalytically active metal, a manganese or vanadium promoter on a support as defined herein. Even more preferred is a Fischer-Tropsch catalyst comprising a cobalt catalytically active metal and a manganese promoter on a support as defined herein.

Typically, the amount of catalytically active metal in the catalyst may range from 1 to 100 parts by weight per 100 parts by weight of support material, preferably from 3 to 50 parts by weight per 100 parts by weight of support material.

Certain compounds having the formula XₐY_{b}O_{c} may be recovered as minerals, for example FeTiO₃. Such minerals are then reduced in size, for example by milling. Preferably submicron particles are obtained. Alternatively they or other compounds having the formula XₐY_{b}O_{c} may be prepared from elements or other compounds, optionally in the presence of water.

One process for preparing a Fischer-Tropsch catalyst support material comprises the steps of:
a) mixing a material comprising at least one element selected from group (i), group (i) consisting of magnesium, calcium, barium, strontium, cerium, vanadium, chromium, manganese, iron, cobalt, nickel, copper, zinc, niobium, ruthenium, rhodium, palladium, cadmium, osmium, iridium, platinum, gold, mercury, tin, lead, lanthanides, and mixtures thereof, especially manganese, vanadium, iron, cobalt, nickel and ruthenium; with a compound comprising at least one element selected from group (ii), group (ii) consisting of silicon, aluminium, titanium, zirconium, cerium, gallium and hafnium, especially silicon, aluminium, titanium and mixtures thereof, more especially titanium;
b) calcining the mixture obtained in step a) at a temperature of 400-2000° C., or reacting in the presence of water at a temperature of up to 20 to 300° C., to form a compound having the formula XₐY_{b}O_{c} wherein X represents the element from the group (i), Y represents a different element from the group (ii), O is oxygen, a and b are, independently, in the range of 1-6 and c is in the range of 1-15.

Preferably the temperature of the calcination in step b is more than 500° C., more preferably above 650° C., and most preferably around 750-850° C. although it may be up to 1200° C.

The effect of the calcination treatment is to remove chemically or physically bonded water such as crystal water, to decompose volatile decomposition products and to convert organic and inorganic compounds to their respective oxides. Another effect of the calcination treatment is the forming of a compound having the formula XₐY_{b}O_{c} from its precursors from group (i) and (ii).

In the presence of water, preferably the reaction is performed at 200-300° C. Preferably the reaction is performed at elevated pressure.

Where more than one element is selected for X, for example X′ and X″, their respective oxides may be mixed from the outset, typically by ball milling to grind the particles to their required size.

The catalytically active metal and the promoter, if present, may be formed with the support material by any suitable treatment, such as dispersing or co-milling. Alternatively, impregnation, kneading and extrusion may be used.

Alternatively the material having the formula XₐY_{b}O_{c} may be recovered as a mineral deposit. Certain compounds having the formula XₐY_{b}O_{c} may be recovered as minerals, for example: V₂TiO₅, V₂Ti₃O₉, MnTiO₃. Alternatively they or other compounds having the formula XₐY_{b}O_{c} may be prepared from elements or other compounds, optionally in the presence of water.

However the present invention also provides a process for making a Fischer-Tropsch catalyst said process comprising the steps of:
a) providing a Fischer-Tropsch catalyst support material consisting of at least 15 wt % of a material having the formula XₐY_{b}O_{c} as described herein;
b) reducing XₐY_{b}O_{c} compound to allow a portion of the metal X to come out of the XₐY_{b}O_{c} compound and function as a catalytically active metal.

Optionally the material having the formula XₐY_{b}O_{c} is prepared by mixing a material comprising at least one of silicon, aluminium, titanium, zirconium, cerium, gallium and hafnium, especially at least one of silicon, aluminium and titanium, more especially titanium with an element chosen from the group defined herein for X; and b) calcining the mixture obtained in step a) at a temperature of over 600° C., or at a temperature of 200-300° C. in the presence of water.

Alternatively the material having the formula XₐY_{b}O_{c} may be recovered as a mineral deposit.

Optionally a vessel may be provided upstream of a Fischer-Tropsch reactor to provide for reduction step c.

Alternatively an active metal may be deposited onto the support by conventional methods such as:
c) contacting said material having the formula XₐY_{b}O_{c} with a solution of a compound of a catalytic metal to obtain a catalyst precursor;
d) drying the catalyst precursor;
e) calcining the catalyst precursor at a temperature in the range of 350 to 750° C., preferably 450-600° C.

The catalyst may also comprise a layer of ceramic ‘glue’ in order to facilitate adherence of the active metal on the support. Suitable ceramic materials include aluminates or silicates.

Preferably the resulting catalyst is the Fischer-Tropsch catalyst as described for earlier aspects of the invention.

Preferably the catalytically active metal compound is a compound of a metal selected from the group consisting of: ruthenium, iron, cobalt, ruthenium and nickel, especially cobalt.

Preferably a compound of a promoter is added along with the compound of the catalytically active metal. The catalytically active metal and the promoter, if present, may be formed with the support material by any suitable treatment, such as dispersing or co-milling. Alternatively, impregnation, kneading and extrusion may be used.

After the calcination steps, the resulting catalyst is usually activated by contacting the catalyst with hydrogen or a hydrogen-containing gas, typically at temperatures of about 200 to 450° C.

Where the mixture in step a is mixing alumina or silica with a metal, preferably the alumina or silica is in the alpha crystalline form. Preferably the silica, if used, is quartzite.

The catalyst may then be used in a Fischer-Tropsch reaction.

Thus the invention also provides the use of a compound as a Fischer-Tropsch catalyst support, at least 15 wt % of the compound consisting of a material of the formula XₐY_{b}O_{c} wherein:

X comprises an element selected from the group consisting of magnesium, calcium, barium, strontium, cerium vanadium, chromium, manganese, iron, cobalt, nickel, copper, zinc, niobium, ruthenium, rhodium, palladium, cadmium, osmium, iridium, platinum, gold, mercury, tin, lead, lanthanides and mixtures thereof, especially manganese, vanadium, iron, cobalt, nickel and ruthenium;

Y comprises a different element to X, Y being selected from the group consisting of silicon, aluminium, titanium, zirconium, hafnium, gallium and cerium and mixtures of these, preferably silicon, aluminium, titanium and mixtures of these, especially titanium;

O is oxygen;

a and b are, independently, in the range of 1-6;

c is in the range of 1-15.

According to a further aspect of the invention, there is provided a process for the production of hydrocarbons from synthesis gas, the process comprising:

converting synthesis gas in a reactor into liquid hydrocarbons, and optionally solid hydrocarbons and optionally liquefied petroleum gas, at elevated temperatures and pressures; using a catalyst as described herein.

The Fischer-Tropsch process is well known to those skilled in the art and involves synthesis of hydrocarbons from syngas, by contacting the syngas at reaction conditions with the Fischer-Tropsch catalyst.

The synthesis gas can be provided by any suitable means, process or arrangement. This includes partial oxidation and/or reforming of a hydrocarbonaceous feedstock as is known in the art.

Typically the synthesis gas is produced by partial oxidation of a hydrocarbonaceous feed. The hydrocarbonaceous feed suitably is methane, natural gas, associated gas or a mixture of C1-4 hydrocarbons. The feed comprises mainly, i.e. more than 90 v/v %, especially more than 94%, C1-4 hydrocarbons, especially comprises at least 60 v/v percent methane, preferably at least 75 percent, more preferably 90 percent. Very suitably natural gas or associated gas is used. As described above, any sulphur in the feedstock is preferably removed or at least minimized.

The partial oxidation of gaseous feedstocks, producing mixtures of especially carbon monoxide and hydrogen, can take place according to various established processes. These processes include the Shell Gasification Process. A comprehensive survey of this process can be found in the Oil and Gas Journal, Sep. 6, 1971, pp 86-90.

The oxygen containing gas for the partial oxidation typically contains at least 95 vol. %, usually at least 98 vol. %, oxygen. Oxygen or oxygen enriched air may be produced via cryogenic techniques, but could also be produced by a membrane based process, e.g. the process as described in WO 93/06041. A gas turbine can provide the power for driving at least one air compressor or separator of the air compression/separating unit. If necessary, an additional compressing unit may be used after the separation process, and the gas turbine in that case may also provide at the (re)start power for this compressor. The compressor, however, may also be started at a later point in time, e.g. after a full start, using steam generated by the catalytic conversion of the synthesis gas into hydrocarbons.

To adjust the H₂/CO ratio in the syngas, carbon dioxide and/or steam may be introduced into the partial oxidation process. Preferably up to 15% volume based on the amount of syngas, preferably up to 8% volume, more preferable up to 4% volume, of either carbon dioxide or steam is added to the feed. Water produced in the hydrocarbon synthesis may be used to generate the steam. As a suitable carbon dioxide source, carbon dioxide from the effluent gasses of the expanding/combustion step may be used. The H₂/CO ratio of the syngas is suitably between 1.5 and 2.3, preferably between 1.6 and 2.0. If desired, (small) additional amounts of hydrogen may be made by steam methane reforming, preferably in combination with the water gas shift reaction. Any carbon monoxide and carbon dioxide produced together with the hydrogen may be used in the gasification and/or hydrocarbon synthesis reaction or recycled to increase the carbon efficiency. Hydrogen from other sources, for example hydrogen itself, may be an option.

The syngas comprising predominantly hydrogen, carbon monoxide and optionally nitrogen, carbon dioxide and/or steam is contacted with a suitable catalyst in the catalytic conversion stage, in which the hydrocarbons are formed. Suitably at least 70 v/v % of the syngas is contacted with the catalyst, preferably at least 80%, more preferably at least 90%, still more preferably all the syngas.

The Fischer-Tropsch synthesis is preferably carried out at a temperature in the range from 125 to 350° C., more preferably 175 to 275° C., most preferably 200 to 260° C. The pressure preferably ranges from 5 to 150 bar abs., more preferably from 5 to 80 bar abs.

The Fischer-Tropsch tail gas may be added to the partial oxidation process.

The Fischer-Tropsch process can be carried out in a slurry phase regime or an ebullating bed regime, wherein the catalyst particles are kept in suspension by an upward superficial gas and/or liquid velocity.

Another regime for carrying out the Fischer-Tropsch process is a fixed bed regime, especially a trickle flow regime. A very suitable reactor is a multitubular fixed bed reactor. In addition, the Fischer-Tropsch process may also be carried out in a fluidized bed process.

Products of the Fischer-Tropsch synthesis may range from methane to heavy paraffin waxes. Preferably, the production of methane is minimized and a substantial portion of the hydrocarbons produced have a carbon chain length of a least 5 carbon atoms. Preferably, the amount of C₅₊ hydrocarbons is at least 60% by weight of the total product, more preferably, at least 70% by weight, even more preferably, at least 80% by weight, most preferably at least 85% by weight.

The hydrocarbons produced in the process are suitably C3-200 hydrocarbons, more suitably C4-150 hydrocarbons, especially C5-100 hydrocarbons, or mixtures thereof. These hydrocarbons or mixtures thereof are liquid or solid at temperatures between 5 and 30° C. (1 bar), especially at about 20° C. (1 bar), and usually are paraffinic of nature, while up to 30 wt %, preferably up to 15 wt %, of either olefins or oxygenated compounds may be present.

Depending on the catalyst and the process conditions used in a Fischer-Tropsch reaction, various proportions of normally gaseous hydrocarbons, normally liquid hydrocarbons and optionally normally solid hydrocarbons are obtained. It is often preferred to obtain a large fraction of normally solid hydrocarbons. These solid hydrocarbons may be obtained up to 90 wt % based on total hydrocarbons, usually between 50 and 80 wt %.

A part may boil above the boiling point range of the so-called middle distillates. The term “middle distillates”, as used herein, is a reference to hydrocarbon mixtures of which the boiling point range corresponds substantially to that of kerosene and gasoil fractions obtained in a conventional atmospheric distillation of crude mineral oil. The boiling point range of middle distillates generally lies within the range of about 150 to about 360° C.

The higher boiling range paraffinic hydrocarbons, if present, may be isolated and subjected to a catalytic hydrocracking step, which is known per se in the art, to yield the desired middle distillates. The catalytic hydro-cracking is carried out by contacting the paraffinic hydrocarbons at elevated temperature and pressure and in the presence of hydrogen with a catalyst containing one or more metals having hydrogenation activity, and supported on a support comprising an acidic function. Suitable hydrocracking catalysts include catalysts comprising metals selected from Groups 6 and 8-10 of the Periodic Table of Elements. Preferably, the hydrocracking catalysts contain one or more noble metals from Groups 8-10. Preferred noble metals are platinum, palladium, rhodium, ruthenium, iridium and osmium. Most preferred catalysts for use in the hydro-cracking stage are those comprising platinum.

The amount of catalytically active noble metal present in the hydrocracking catalyst may vary within wide limits and is typically in the range of from about 0.05 to about 5 parts by weight per 100 parts by weight of the support material. The amount of non-noble metal present is preferably 5-60%, preferably 10-50%.

Suitable conditions for the catalytic hydrocracking are known in the art. Typically, the hydrocracking is effected at a temperature in the range of from about 175 to 400° C. Typical hydrogen partial pressures applied in the hydrocracking process are in the range of from 10 to 250 bar.

The product of the hydrocarbon synthesis and consequent hydrocracking suitably comprises mainly normally liquid hydrocarbons, beside water and normally gaseous hydrocarbons. By selecting the catalyst and the process conditions in such a way that especially normally liquid hydrocarbons are obtained, the product obtained (“syncrude”) may be transported in the liquid form or be mixed with any stream of crude oil without creating any problems as to solidification and or crystallization of the mixture. It is observed in this respect that the production of heavy hydrocarbons, comprising large amounts of solid wax, are less suitable for mixing with crude oil while transport in the liquid form has to be done at elevated temperatures, which is less desired.

Thus according to a further aspect of the invention, there is provided hydrocarbon products synthesised by a Fischer-Tropsch reaction and catalysed by a catalyst as described herein.

The hydrocarbon products may have undergone the steps of hydroprocessing, preferably hydrogenation, hydroisomerization and/or hydrocracking.

The hydrocarbon products may be fuels, preferably naphtha, kerosene or gasoil, a waxy raffinate or a base oil.

Any percentage mentioned in this description is calculated on total weight or volume of the composition, unless indicated differently. When not mentioned, percentages are considered to be weight percentages. Pressures are indicated in bar absolute, unless indicated differently.

An embodiment of the present invention will now be described by way of example only.

### EXPERIMENTAL

### Experiment 1

A catalyst support comprising cobalt titanate was prepared in accordance with the present invention. At 400° C. there was no reduction of the cobalt in this catalyst indicating that the cobalt present is in the form of cobalt titanate. This catalyst support was shown not to be active under Fischer-Tropsch conditions which also demonstrates that the cobalt present is in the form of cobalt titanate.

In contrast a traditional catalyst outside the scope of the present invention was prepared using Co(OH) and TiO₂. After calcination 29% CO₃O₄ was present. In this example all the material was reduced at 280° C. to active cobalt.

### Experiment 2

XRD analysis of a catalyst support in accordance with the present invention showed the presence of 16% CO₂TiO₄ with a 022 crystal plane size of 12 nm.

### Experiment 3

Nano crystalline MnTiO₃ was prepared by calcining at 900° C. a mixture of TiO₂ and Mn(OH)₂. The obtained material was mixed with Co(OH)₂ and shaped into extrudates via co-milling and extrusion, followed by drying and calcination at 600 C. After reduction the catalyst was ready to be used in a Fischer Tropsch reaction.

What is claimed:

1. A Fischer-Tropsch catalyst support comprising at least 15 wt % of a material having the formula XₐY_{b}O_{c} wherein:
X comprises an element selected from the group consisting of magnesium, calcium, barium, strontium, cerium, vanadium, chromium, manganese, iron, cobalt, nickel, copper, zinc, niobium, ruthenium, rhodium, palladium, cadmium, osmium, iridium, platinum, gold, mercury, tin, lead, lanthanides, and mixtures thereof;
Y comprises a different element to X, Y selected from the group consisting of silicon, aluminium, titanium, zirconium, cerium, hafnium, gallium, and mixtures thereof;
O is oxygen;
a and b are, independently, in the range of 1-6;
c is in the range of 1-15.

2. A catalyst support as claimed in claim 1, wherein XₐY_{b}O_{c} has a perovskite-type structure.

3. A catalyst support as claimed in claim 1, having the general formula (XO)ᵢ(YO₂)ᵢᵢ wherein (i) and (ii) are arbitrary constants in the range of 1-5.

4. A catalyst support as claimed in claim 1, wherein at least 30 wt % of the catalyst support comprises XₐY_{b}O_{c}.

5. A catalyst support as claimed in claim 1, wherein at least 80 wt % of the catalyst support comprises XₐY_{b}O_{c}.

6. A catalyst support as claimed in claim 1, wherein at least 95 wt % of the catalyst support comprises XₐY_{b}O_{c}.

7. A catalyst support as claimed in claim 1, further comprising (XX′)ₐY_{b}O_{c} wherein X, Y, a, b, O and c are as previously defined and X′ is a different element to X and is selected from the group previously defined for X.

8. A catalyst support as claimed in claim 1, further comprising X″ₐY_{b}O_{c} wherein a, Y, b, O and c are as previously defined and X″ is a different element to X and is selected from the group previously defined for X.

9. A catalyst support as claimed in claim 1, wherein the element X is selected from the group consisting of manganese, vanadium, magnesium, calcium, barium, strontium, cerium, cobalt, iron, ruthenium, chromium, tin, lead, palladium, lanthanides, lanthanides, nickel, and mixtures thereof.

10. A catalyst support as claimed in claim 9, wherein the material having the formula XₐY_{b}O_{c} is selected from the group consisting of CoTiO₃, CO₂TiO₄, FeSiO₃, NiSiO₃ and FeAl₂O₅, or mixtures thereof.

11. A catalyst support as claimed in claim 9, wherein the material having the formula XₐY_{b}O_{c} is selected from the group consisting of MnTiO₃, MnAl₂O₄, MnSiO₃, Mn₂TiO₄, MnTi₂O₅ and MnTi₃O₇.

12. A Fischer-Tropsch catalyst comprising:
a catalytically active metal or precursor therefor, the catalytically active metal being selected from the group consisting of ruthenium, iron, cobalt and nickel, or combinations thereof, preferably cobalt;
a Fischer-Tropsch catalyst support as claimed in claim 1, said catalyst support comprising as element X the same metal as the catalytically active metal.

13. A process for the production of hydrocarbons from synthesis gas, the process comprising converting synthesis gas in a reactor into liquid hydrocarbons, at elevated temperatures and pressures using a catalyst as claimed in claim 12.

14. A process for preparing a Fischer-Tropsch catalyst said process comprising the steps of:
a) providing a Fischer-Tropsch catalyst support material comprising at least 15% of a material having the formula XₐY_{b}O_{c} wherein
X comprises an element selected from the group consisting of magnesium, calcium, barium, strontium, cerium, vanadium, chromium, manganese, iron, cobalt, nickel, copper, zinc, niobium, ruthenium, rhodium, palladium, cadmium, osmium, iridium, platinum, gold, mercury, tin, lead, lanthanides, and mixtures thereof;
Y comprises a different element to X, Y selected from the group consisting of silicon, aluminium, titanium, zirconium, cerium, hafnium, gallium, and mixtures of these;
O is oxygen;
a and b are, independently, in the range of 1-6;
c is in the range of 1-15;
b) reducing XₐY_{b}O_{c} compound to allow a portion of the metal X to come out of the XₐY_{b}O_{c} compound and function as a catalytically active metal or promoter.
